(19) United States
(12) Patent Application Publication
(10) Pub. No.: US 2017/0219503 A1
(43) Pub. Date: Aug. 3, 2017

(54) APPARATUS AND METHOD FOR X-RAY PHASE CONTRAST IMAGING

(71) Applicant: University of Massachusetts, Boston, MA (US)

(72) Inventors: Srinivasan Vedantham, Holden, MA (US); Andrew Karellas, Grafton, MA (US)

(21) Appl. No.: 15/385,518

(22) Filed: Dec. 20, 2016

Related U.S. Application Data
(63) Continuation of application No. 14/379,041, filed on Aug. 15, 2014, now abandoned.
(60) Provisional application No. 61/602,923, filed on Feb. 24, 2012.

(51) Int. Cl. G01N 23/20; A61B 6/00; G21K 1/06; G01N 23/04

(52) U.S. Cl. CPC G01N 23/20075; G01N 23/04; A61B 6/484; G21K 1/067; A61B 6/4035; A61B 6/4291; G01N 2223/6126

(57) ABSTRACT

An x-ray phase contrast imaging apparatus and method of operating the same. The apparatus passes x-rays generated by an x-ray source through, in succession, a source grating, an object of interest, a phase grating, and an analyzer grating. The x-ray source, the source grating, the phase grating, and the analyzer grating move as a single entity relative to an object of interest. The phase grating and the analyzer grating remain in fixed relative location and fixed relative orientation with respect to one another. The detected x-rays are converted to a time sequence of electrical signals. In some cases, the apparatus is controlled, and the electrical signals are analyzed by, by a general purpose programmable computer provided with instructions recorded on a machine readable medium. One or more x-ray phase contrast images of the object of interest are generated, and can be recorded or displayed.

### PRIORITY CLAIMS AND RELATED PATENT APPLICATIONS

This application is the national phase of PCT/US13/26530, filed Feb. 15, 2013, which claims the benefit of priority from U.S. Provisional Application Ser. No. 61/602,923, filed on Feb. 24, 2012, the entire content of which is incorporated herein by reference in its entirety.

### STATEMENT OF FEDERALLY FUNDED RESEARCH

This invention was made with government support under grant numbers. R21 CA134128 and R01 CA128906 awarded by the National Institutes of Health. The government has certain rights in the invention.

### TECHNICAL FIELD OF THE INVENTION

The invention relates to x-ray imaging in general and particularly to x-ray imaging that employs phase contrast imaging methods.

### BACKGROUND OF THE INVENTION

Clinical x-ray imaging techniques provide image contrast between the various tissues that comprise the anatomy being imaged through absorption contrast that is related to the x-ray attenuation properties of the tissues. These include modalities such as radiography, mammography, tomosynthesis, and computed tomography (CT). However, the imaged tissue can also be characterized by its refractive index. When x-rays propagate through an object, the associated phase and intensity changes can be represented by the object's complex index of refraction, n=1−δ+iβ, where δis the refractive index decrement that is responsible for the phase shift, and β is the absorption index. β is related to the mass attenuation coefficient

$${μ}_{m} = \frac{4 πβ}{λρ}$$

and is the basis for image contrast in conventional x-ray imaging including mammography, radiography, tomosynthesis and CT. In the above equation, λ is the wavelength of the x-rays and ρ is the density of the object being imaged. At energy (and hence wavelength) levels away from the absorption edge of the object being imaged, the refractive index δ can be calculated as

$$δ = \frac{{r}_{0} {λ}^{2} {ρ}_{e}}{2 π} ,$$

where r₀ is the classical electron radius and ρₑ is the electron density of the object being imaged. Since the early 1990's, phase contrast x-ray imaging has being actively investigated. Broadly, based on the imaging geometry and its hardware implementation, phase-sensitive imaging techniques can be classified as (i) inline phase-propagation x-ray imaging; (ii) diffraction enhanced imaging; and, (iii) interferometry. Among these techniques, Talbot interferometry is widely considered as the technique that is best suited for clinical adaptation as it can be performed in a relatively short exam duration using conventional x-ray sources, i.e., x-ray tubes used in current clinical systems. (See W. H. F. Talbot, “Facts relating to optical science, No. IV”, Philosophical Magazine 9, 401 (1836).) A key enabling feature that allows the use of a conventional x-ray tube is the use of a source grating (e.g., a thin plate with parallel trenches or strips with alternating high and low attenuation) that provides multiple individually coherent sources. (C. Kottler, F. Pfeiffer, O. Bunk, C. Grunzweig and C. David, “Grating interferometer based scanning setup for hard X-ray phase contrast imaging”, Rev Sci Instrum 78 (4), 043710 (2007); F. Pfeiffer, T. Weitkamp, O. Bunk and C. David, “Phase retrieval and differential phase-contrast imaging with low-brilliance X-ray sources”, Nat Phys 2 (4), 258-261 (2006).)

While the principle of Talbot-interferometry is well known, Pfeiffer and his colleagues pioneered a technique that is practical for some radiographic imaging tasks. (See M. Engelhardt, C. Kottler, O. Bunk, C. David, C. Schroer, J. Baumann, M. Schuster and F. Pfeiffer, “The fractional Talbot effect in differential x-ray phase-contrast imaging for extended and polychromatic x-ray sources”, J Microsc 232 (1), 145-157 (2008); C. Kottler, C. David, F. Pfeiffer and O. Bunk, “A two-directional approach for grating based differential phase contrast imaging using hard x-rays”, Opt Express 15 (3), 1175-1181 (2007); T. Weitkamp, A. Diaz, C. David, F. Pfeiffer, M. Stampanoni, P. Cloetens and E. Ziegler, “X-ray phase imaging with a grating interferometer”, Optics Express 13 (16), 6296-6304 (2005); F. Pfeiffer, M. Bech, O. Bunk, P. Kraft, E. F. Eikenberry, C. Bronnimann, C. Grunzweig and C. David, “Hard-X-ray dark-field imaging using a grating interferometer”, Nat Mater 7 (2), 134-137 (2008).)

In their approach, (FIG. 1, Prior Art), a source grating, G0, with pitch P₀ allows for “individually coherent (but mutually incoherent)” beams to pass through its trenches from a conventional x-ray tube of focal spot size, w. Typically, G0 is a gold-filled source grating. The beams pass through an object of interest. After the object, a phase-shifting grating, G1, with pitch P₁ is placed in the beam (working as a “beam splitter”). For the purpose of the present discussion, the pitch is defined as the center-to-center spacing of adjacent trenches of a grating. The resulting interference pattern creates a “self-image” of the grating G1 at fractional Talbot distances. (See L. Rayleigh, “On copying diffraction-gratings, and on some phenomenon connected therewith”, Philosophical Magazine 11, 196 (1881).)

The x-ray beam deflection by an object shifts the interference pattern, i.e., the relative positions of its minima and maxima, along the x direction. This shift in interference pattern is proportional to the derivative ∂φ/∂x of the x-ray wave-field φ in the direction perpendicular to the grating trenches, which are oriented along the y direction. Since the shift is small, it is difficult to directly image the fine structures and the shifts in the interference pattern with current detectors. By introducing an analyzer (absorber) grating, G2, with periodicity identical to the interference pattern, Moire patterns are generated with much larger periodicity that can be detected by current detectors. G2 is a gold-filled analyzer grating, which is translated relative to grating G1 as indicated by the bidirectional arrow. Phase stepping images are acquired at each position during translation. To experimentally measure the phase gradient, the analyzer grating G2 needs to be shifted along the x direction, by a fraction of its pitch P₂, a procedure often referred to as “phase stepping.” For each detector pixel, the phase stepping signal is large if the intensity maxima of the interference pattern coincide with the gaps of G2, and the signal is weak if the intensity maxima coincide with the absorber bars of G2. The acquired signal series per pixel takes the form of a periodic function. It is thus possible to obtain three pieces of information for each pixel: (i) the object absorption (attenuation) from the intensity averaged over all phase steps; (ii) the phase gradient (proportional to the lateral shift of the interference pattern) from the fringe phase of the phase stepping curve; and, (iii) the fringe visibility, from the amplitude of the intensity modulation during the phase stepping and can be used for x-ray dark-field imaging

U.S. Pat. No. 7,492,871 B2 dated Feb. 17, 2009 is said to disclose a focus/detector system of an x-ray apparatus for generating phase contrast recordings where the detector elements are formed by a multiplicity of scintillation strips that serve the dual purpose of an analyzer grating and a detector.

U.S. Pat. No. 7,693,256 B2 dated Apr. 6, 2010 is said to disclose a phase contrast x-ray imaging system that is capable of stereoscopic imaging and comprises a stereoscopic radiation head.

U.S. Patent Publication No. 2010/0322380 A1 dated Dec. 23, 2010 is said to disclose a detector for x-ray phase contrast imaging that comprise a phase grating and at least two analyzer gratings to record the differential phase information over a macroscopic pixel.

U.S. Pat. No. 7,983,381 B2 dated Jul. 19, 2011 is said to disclose an x-ray CT system for x-ray phase contrast and/or x-ray dark field imaging where the object to be imaged is interposed between the phase and analyzer grating.

U.S. Pat. No. 8,009,796 B2 dated Aug. 30, 2011 is said to disclose an x-ray CT system to generate tomographic phase contrast or dark field exposures that comprise multiple modules each comprising a phase grating, an analyzer grating and a detector, where the distance between the gratings within each module is adapted to the divergence (fan angle) of the x-ray beam.

U.S. Pat. No. 8,041,004 B2 dated Oct. 18, 2011 is said to disclose an x-ray interferometer for phase contrast imaging that comprises at least one line detector and the object is moved to provide the differential phase contrast images.

There is a need for improved x-ray systems and methods for generating x-ray phase contrast images.

### SUMMARY OF THE INVENTION

According to one aspect, the invention features an x-ray phase contrast imaging apparatus. The x-ray phase contrast imaging apparatus comprises an x-ray source configured to provide x-ray illumination at an exit port thereof; a source grating configured to receive the x-ray illumination at a source grating entrance port and configured to provide a plurality of x-ray beams at a source grating exit port; a phase grating having a plurality of phase grating elements, the phase grating situated at a distance l from the source grating, the phase grating configured to receive x-rays at a phase grating entrance port and to provide x-rays at a phase grating exit port; a analyzer grating having a plurality of analyzer grating elements, the analyzer grating situated at a distance d from the phase grating, the phase grating and the analyzer grating having a fixed location and a fixed orientation relative to each other, the analyzer grating configured to receive x-rays at a analyzer grating entrance port and to provide x-rays at a analyzer grating exit port; the x-ray source, the source grating, the phase grating, and the analyzer grating configured to move as a single entity relative to an object of interest; an x-ray sensitive detector positioned so as to receive x-rays generated by the x-ray source after the x-rays have passed sequentially through the source grating, through the object of interest, through the phase grating and through the analyzer grating, the x-ray-sensitive detector having at least one output terminal configured to provide electrical signals representative of the received x-rays; a controller configured to control the motion of the x-ray source, the source grating, the phase grating, and the analyzer grating relative to the object of interest as a function of time, and configured to control the x-ray source and the x-ray sensitive detector as a function of time; and an analyzer module configured to receive and record the electrical signals representative of the received x-rays as a function of time, configured to manipulate the received electrical signals with respect to time, configured to generate a phase contrast image of at least a portion of the object of interest the from received electrical signals, and configured to perform at least one action selected from the group of actions consisting of recording the x-ray phase contrast image, transmitting the x-ray phase contrast image to a data handling system, and displaying the x-ray phase contrast image to a user.

In one embodiment, the x-ray source is a conventional x-ray tube with an x-ray focal spot.

In another embodiment, the x-ray source is an x-ray source selected from the group of x-ray sources consisting of a hot filament x-ray source and a field emission x-ray source.

In yet another embodiment, the apparatus further comprises an object support configured to support the object of interest.

In still another embodiment, the apparatus further comprises a compression paddle.

In a further embodiment, the x-ray sensitive detector is selected from the group of x-ray sensitive detectors consisting of a one dimensional array of x-ray sensitive pixels and a two dimensional array of x-ray sensitive pixels.

In yet a further embodiment, the analyzer module is a general purpose programmable computer provided with instructions recorded on a machine readable medium.

In an additional embodiment, the controller and the analyzer module are each part of a single general purpose programmable computer provided with instructions recorded on a machine readable medium.

In one more embodiment, the phase grating having a plurality of phase grating elements and the analyzer grating having a plurality of analyzer grating elements are configured such that a first phase shift is provided between a first of the plurality of phase grating elements and a first of the plurality of analyzer grating elements, a second phase shift is provided between a second of the plurality of phase grating elements and a second of the plurality of analyzer grating elements, and a third phase shift is provided by a third of the plurality of phase grating elements and a third of the plurality of analyzer grating elements. The phase shifts, which should cover a range of 2π radians, can be measured as a time sequence. While the range of phase shift we mention can be understood as having a range from 0 to 2π, it is also possible to use the range from −π to π radians, or in general, any range from angle R radians to R+2π radians. One needs to make at the minimum three measurements to meet the Nyquist sampling criterion. For example, for a sine wave covering the angular range [0,2π], the amplitudes at 0, π, and 2π will all be zero. Thus, it may be sufficient to measure at one of these points. The other two points one could in principle measure are π/2 and 3π/2 that correspond to the maximum and minimum amplitudes of the sine wave. However, as long as the angular relationship between the three measurements are known, and they are not all separated by exactly π radians, one can always determine the characteristics of the sine wave from the three measurements. The order in which the phase shift is measured is unimportant. As long as one knows which phase/analyzer grating combination is providing the measurement, one can determine the specific phase shift. Increasing the number of measured phase shifts can improve the determination of the characteristics of the sine wave.

In yet one more embodiment, the source grating is configured to provide a plurality of x-ray beams that individually exhibit spatial coherence at the source grating exit port.

According to another aspect, the invention relates to a method of making an x-ray phase contrast image of an object of interest. The method comprises the steps of passing x-rays generated by an x-ray source through, in succession, a source grating, an object of interest, a phase grating, and a analyzer grating while causing the x-ray source, the source grating, the phase grating, and the analyzer grating to move as a single entity relative to an object of interest, the phase grating and the analyzer grating remaining in fixed relative location and fixed relative orientation with respect to one another; detecting transmitted x-rays with an x-ray sensitive detector, the x-ray sensitive detector providing electrical signals representative of the detected x-rays as output signals; analyzing the electrical signals representative of the detected x-rays as a function of time to generate an x-ray phase contrast image of the object of interest; and performing at least one action selected from the group of actions consisting of recording the x-ray phase contrast image, transmitting the x-ray phase contrast image to a data handling system, and displaying the x-ray phase contrast image to a user.

The foregoing and other objects, aspects, features, and advantages of the invention will become more apparent from the following description and from the claims.

### BRIEF DESCRIPTION OF THE DRAWINGS

The objects and features of the invention can be better understood with reference to the drawings described below, and the claims. The drawings are not necessarily to scale, emphasis instead generally being placed upon illustrating the principles of the invention. In the drawings, like numerals are used to indicate like parts throughout the various views.

FIG. 1 is a schematic diagram of a prior art Talbot-grating based differential phase contrast imaging system.

FIG. 2 is a schematic diagram of an embodiment of an apparatus according to the invention.

FIG. 3A, FIG. 3B, FIG. 3C, and FIG. 3D are schematic diagrams showing the scanning process during an examination of an object of interest.

FIG. 4 shows an embodiment having four sets of phase gratings G1 and analyzer gratings G2.

FIG. 5A is a schematic diagram that illustrates a method for generating differential phase contrast images at a first time t1.

FIG. 5B is a schematic diagram that illustrates a method for generating differential phase contrast images at a second time t2.

FIG. 6A is a schematic diagram that illustrates an embodiment of an apparatus with detector sub-assembly comprising multiple line detectors, each of width, Xd along the scan direction and pixel spacing Py in the direction orthogonal to the scan and spaced td apart. Gratings G1 and G2 are not shown for clarity.

FIG. 6B is a schematic diagram that illustrates a method for obtaining differential phase contrast images, showing the relative positions of the detector sub-assembly at two times, t1 (detector positions shown in solid lines) and t2 (detector positions shown in broken lines). Gratings G1 and G2 are not shown for clarity in FIG. 6B.

FIG. 7A is a schematic diagram that illustrates gratings G1 and G2 that are oriented such that the scan lines are parallel to the scan direction, and in which the gratings G2 and G1 are slightly tilted with respect to one another.

FIG. 7B is a schematic diagram similar to FIG. 7A, in which multiple line detectors are used instead of a two-dimensional pixel array detector.

FIG. 8 is a schematic diagram that illustrates the components of an apparatus according to principles of the invention and the interactions among the components.

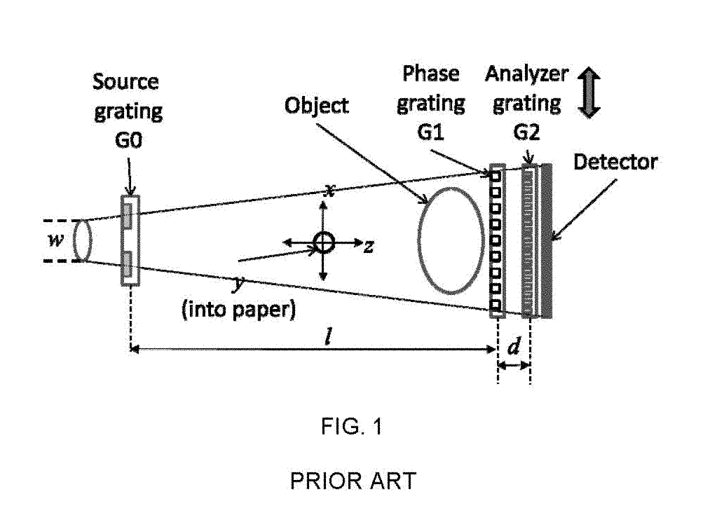

### DETAILED DESCRIPTION OF THE INVENTION

The invention pertains to an apparatus and method for x-ray phase contrast imaging that uses a scanning approach for the x-ray source, source grating, phase grating and detector grating, with the object being imaged stationary. Importantly, the apparatus and method does not require movement of the phase or detector grating relative to each other, often referred to as phase-stepping, which is typically a fraction of the period of phase grating, P₂. This alleviates the need for high-precision stepping mechanism. While multiple preferred embodiments are provided, the common feature of the apparatus is the use of a scanning interferometer comprising an x-ray source, source grating, phase grating and detector grating that scans the object, with the object being imaged stationary and interposed between the source and phase gratings.

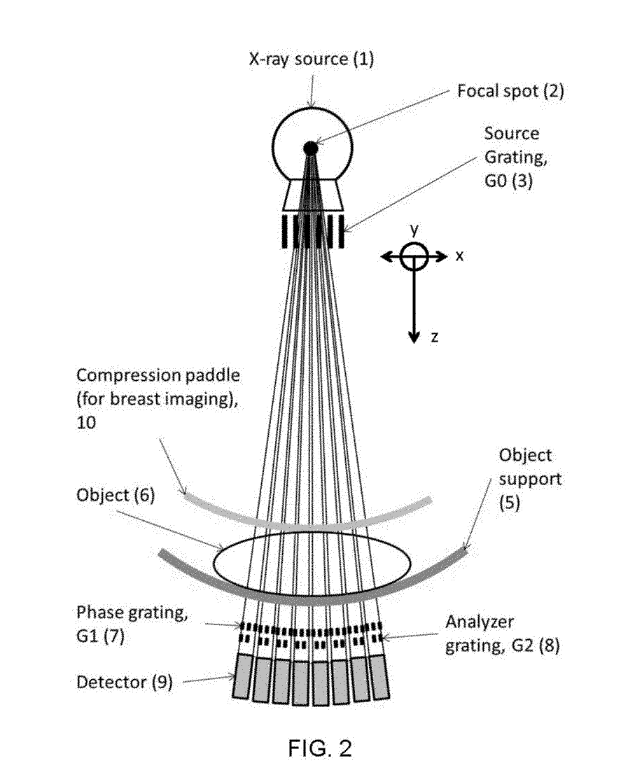

The apparatus of the invention is shown in FIG. 2. The apparatus comprises an x-ray source (1). In some embodiments, the x-ray source 1 is a conventional x-ray tube with an x-ray focal spot (2). In other embodiments the x-ray source 1 is a hot filament type tube or the newer types of compact field emission type tube. A source grating G0 (3) is positioned close to the exit port of the x-ray source. The source grating G0 receives the x-rays generated by the x-ray source and provides a plurality of x-ray beams that are individually coherent but mutually incoherent. An object support (5) is optionally provided, if necessary to support the object (6) being imaged. The object 6 is interposed between the source grating G0 (3) and the phase grating G1 (7). The phase grating G1 is located at a distance l from the source grating G0 (3). An analyzer grating G2 (8) is located at a distance d from the phase grating G1 (7). The locations and orientations of the phase grating G1 and the analyzer grating G2 are fixed with respect to each other. The phase grating G1 and the analyzer grating G2 do not move relative to each other during the operation of the apparatus. An x-ray sensitive detector (9) is located as close as possible to the said phase grating G2 (8). Additionally, the apparatus may include an optional compression paddle (10) for breast imaging.

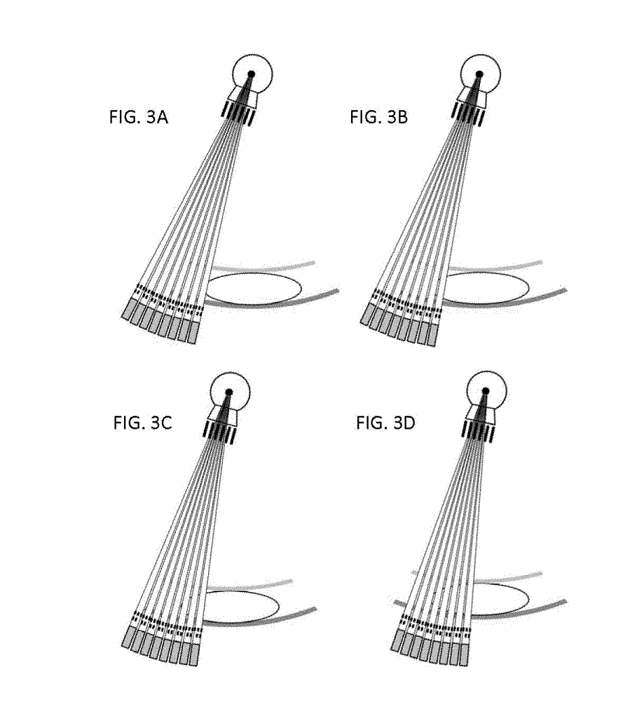

The grating lines of G0, G1 and G2 are oriented such that they intersect the x-ray beam from the x-ray source to the detector. The detector can be either an energy-integrating detector or a photon-counting detector. The imaging geometry is selected such that the conditions for Talbot interferometry are satisfied. These conditions include the periodicity of each of the gratings, their depth and choice of material, the distances l and d for the given design x-ray photon energy, and the wavelength λ. The preferred embodiments describe variations in the location and the orientation of the gratings relative to one another, and the grating structure. A feature of the method for obtaining differential phase contrast images is the synchronization of the scanning movement of the interferometer with the detector readout, so that the differential phase contrast images can be obtained with the desired pixel spacing. This is illustrated in FIG. 3A, FIG. 3B, FIG. 3C and FIG. 3D (which together will be referred to as FIG. 3).

Referring to FIG. 3, the apparatus scans in a given direction, which is shown as a left to right scan for the purposes of illustration in FIG. 3. A scan in the right to left direction is also possible. During the scan, the x-ray source and its x-ray focal spot, the source grating, the phase grating, the analyzer grating and the detector assembly move in unison, while the object being examined and any support that may be provided remain stationary. In the example of breast imaging, the breast, the object support if present, and the compression paddle for breast imaging if present all remain stationary. Herein we will refer to the components that move during the scan of the object as the scanning assembly. The scanning assembly comprises the x-ray source and its x-ray focal spot, the source grating, the phase grating, the analyzer grating and the detector assembly. The detector readout is synchronized with the scanning movement of the scan assembly, so that the object can be sampled and the differential phase contrast images can be acquired with the desired pixel spacing. In FIG. 3, four positions during the scanning movement of the scan assembly traversing left to right are shown for purposes of illustration in FIG. 3A, FIG. 3B, FIG. 3C and FIG. 3D.

### First Exemplary Embodiment

In this embodiment, the grating lines of all three gratings (G0, G1 and G2) are oriented orthogonal to the x-ray beam scan direction. This orientation of the gratings is illustrated in FIG. 2, and in FIG. 3A, FIG. 3B, FIG. 3C and FIG. 3D. A feature of the apparatus described in this embodiment is that the detector is sub-divided into N detector sub-assemblies. FIG. 2 shows an example having 8 detector sub-assemblies. Each detector sub-assembly having a corresponding phase grating (G1) and analyzer grating (G2). The phase grating (G1) and the analyzer grating (G2) are progressively displaced with respect to each other across the N detector sub-assemblies. The relative displacement of phase grating G1 and analyzer grating G2 is such that the over the N detector sub-assemblies measurements covering a phase shift of 2π is achieved. Alternatively stated, each detector sub-assembly provides a measure corresponding to a specific phase shift, and when measurements are performed over N detector sub-assemblies discrete phase shifts covering 2π are measured.

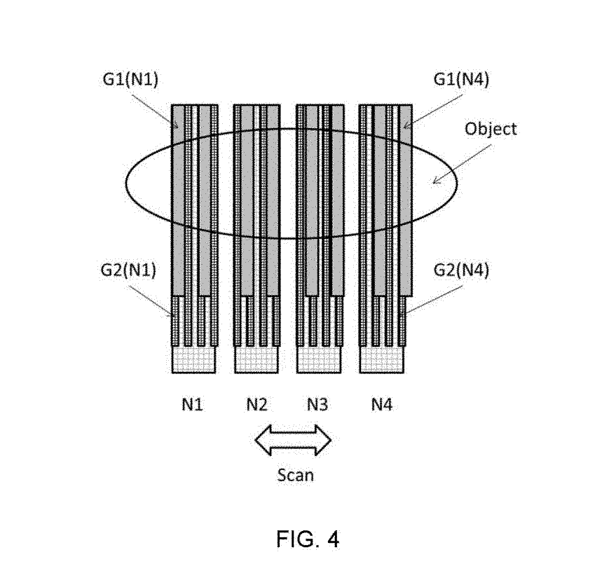

FIG. 4 shows an embodiment having four sets of phase gratings G1 and analyzer gratings G2. When viewed from the x-ray focal spot towards the detector, i.e., along the z-direction shown in FIG. 2, each detector sub-assembly (of which 4 are shown and are labeled N1 through N4, respectively) corresponds to a phase grating (labeled G1(N1) through G1(N4)) and an analyzer grating (labeled G2(N1) through G2(N4)). Across the four detector assemblies shown, the analyzer gratings G2(N1) through G2(N4) are progressively shifted with respect to the corresponding phase gratings G1(N1) through G1(N4). For purposes of illustration and for ease of viewing, the phase gratings G1 are shown shorter than the detector sub-assemblies in the vertical direction, and the analyzer gratings G2 are shown shorter than the phase gratings G1. In the apparatus, the phase gratings G1 and the analyzer gratings G2 are expected to extend vertically so that the entire detector sub-assembly is covered. In some embodiments, each of the detector sub-assembly may contain a plurality of line detectors each with a plurality of pixels or may contain a two-dimensional array detector with a plurality of pixels oriented in two-dimensions. The method for acquiring differential phase contrast images is based on synchronization between readout of each detector sub-assembly and the scanning motion. At a given time t1, each detector sub-assembly provides a measure corresponding to specific phase shift of the region of the object imaged by that detector sub-assembly. At a different time t2, the scan assembly samples the same region of the object that was previously imaged at time instance t1 at a different phase shift. Thus after N detector sub-assemblies have sampled the same region of the object, the differential phase contrast images can be retrieved with appropriate mathematical algorithms.

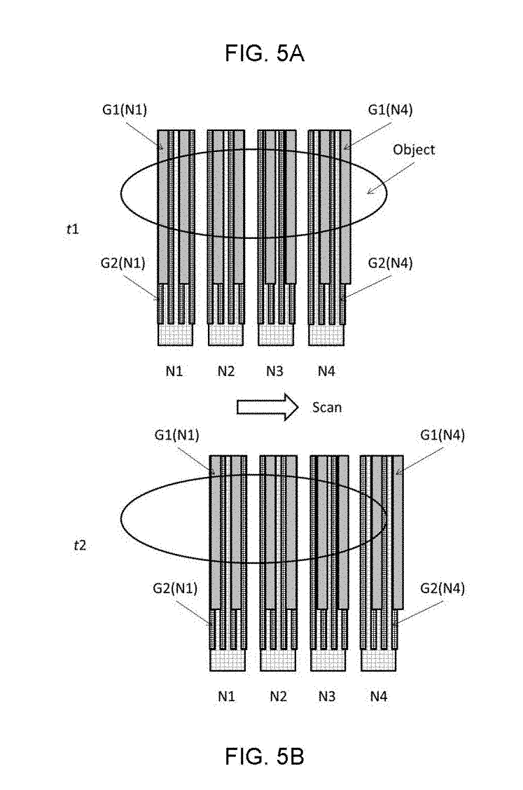

This method is illustrated in FIG. 5A and FIG. 5B. FIG. 5A is a schematic diagram that illustrates a method for generating differential phase contrast images at a first time t1. FIG. 5B is a schematic diagram that illustrates a method for generating differential phase contrast images at a second time t2.

A mathematical description of the processing of data is now presented. In an embodiment in which that N detector sub-assemblies are used and P₂ is the pitch of the analyzer grating, then across the N detector sub-assemblies, the analyzer grating is shifted by an amount

$${X}_{s} = \frac{{iP}_{2}}{N} ,$$

where i varies from 1 to N, e.g., i=1, 2,. . . N. Alternatively stated, the shift between the phase and analyzer grating for any detector sub-assembly Nⁱ is

$$\frac{{iP}_{2}}{N} .$$

Thus at a time t1 if the sub-assembly Nⁱ images a region of the object, the measured phase shift corresponds to a grating shift (between phase and analyzer grating) of

$$\frac{{P}_{2}}{N} .$$

Subsequently, at a different time t2 if the sub-assembly N² images the same region of the object, the measured phase shift corresponds to a grating shift (between phase and analyzer grating) of

$$\frac{2 {P}_{2}}{N} .$$

When all N detector sub-assemblies have imaged the same region of the object, the complete dataset corresponding to all phase shifts between the phase and analyzer grating has been obtained. If I¹(x, y) represents the image recorded at time t1 by detector sub-assembly N¹, then applying the Fourier series expansion, the image corresponds to

$${I}^{1} ( x , y ) = {I}_{0} ( x , y ) + {I}_{1} cos [ \frac{2 π}{M} + φ ( x , y ) ] .$$

Generalizing for any i, for the same region of the object imaged by detector sub-assembly Nⁱ, the image corresponds to

$${I}^{i} ( x , y ) = {I}_{0} ( x , y ) + {I}_{1} cos [ \frac{2 π i}{M} + φ ( x , y ) ] .$$

In the above equation, I₀(x,y) corresponds to the attenuation image (equivalent to a standard radiographic image) and φ(x,y) corresponds to the differential phase contrast image. The differential phase contrast image φ(x,y) can be recovered from the time series image data that corresponds to the same region of the object imaged by all of the N detector sub-assemblies by employing the Fourier transform and can be computed as:

$$φ ( x , y ) = {tan}^{- 1} [ - \frac{{\sum }_{i = 1}^{N} {I}^{i} ( x , y ) sin ( \frac{2 π i}{N} )}{{\sum }_{i = 1}^{N} {I}^{i} ( x , y ) cos ( \frac{2 π i}{N} )} ] .$$

Depending upon the type of detector used, the image Iⁱ(x,y) is expected to be proportional to the number of x-ray photons incident on a pixel in case of photon counting detectors or is expected to be proportion to the product of the number of x-ray photons and its energy incident on a pixel in case of energy integrating detectors. Each pixel may be square or rectangular. In various embodiments, the pixel dimension ranges between 30 and 250 microns, depending on the desired resolution and the imaging application. The directionality of the scan (left to right or right to left) does not matter as all of the desired phase shift measurements are obtained as a time series by the N detector sub-assemblies. In a preferred embodiment, constant angular or scan velocity of the detector assembly is maintained. However, it is not necessary to maintain constant angular or scan velocity as long as the time at which the same region of the object is imaged by each detector sub-assembly is known. Further, the method allows for obtaining the phase contrast image (and not just the differential phase contrast image) by integrating the differential phase contrast image along the direction of the scan, provided the scan covers the entire object.

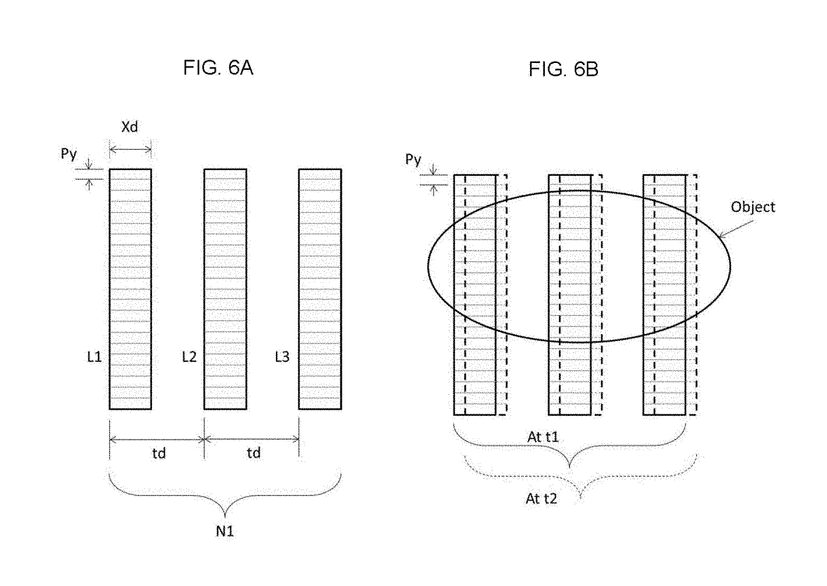

FIG. 6A is a schematic diagram that illustrates an embodiment of an apparatus with detector sub-assembly comprising multiple line detectors, each of width, Xd along the scan direction and pixel spacing Py in the direction orthogonal to the scan and spaced td apart. Gratings G1 and G2 are not shown for clarity. The method for obtaining differential phase contrast images is also described. Although Xd can be larger than Py, by synchronizing the readout of each line detector with the scanning motion the object can be sampled with the same spacing as Py. Gratings G1 and G2 are not shown for clarity in FIG. 6A. Multiple such detector assemblies, each measuring a different phase shift can be used to obtain the differential phase contrast images.

FIG. 6B is a schematic diagram that illustrates a method for obtaining differential phase contrast images, showing the relative positions of the detector sub-assembly at two times, t1 (detector positions shown in solid lines) and t2 (detector positions shown in broken lines). Gratings G1 and G2 are not shown for clarity in FIG. 6B.

### Second Exemplary Embodiment

In this embodiment, the grating lines of all three gratings (G0, G1 and G2) are oriented parallel to the scan direction. The analyzer grating G2 is tilted by a small angle with respect to the phase grating G1.

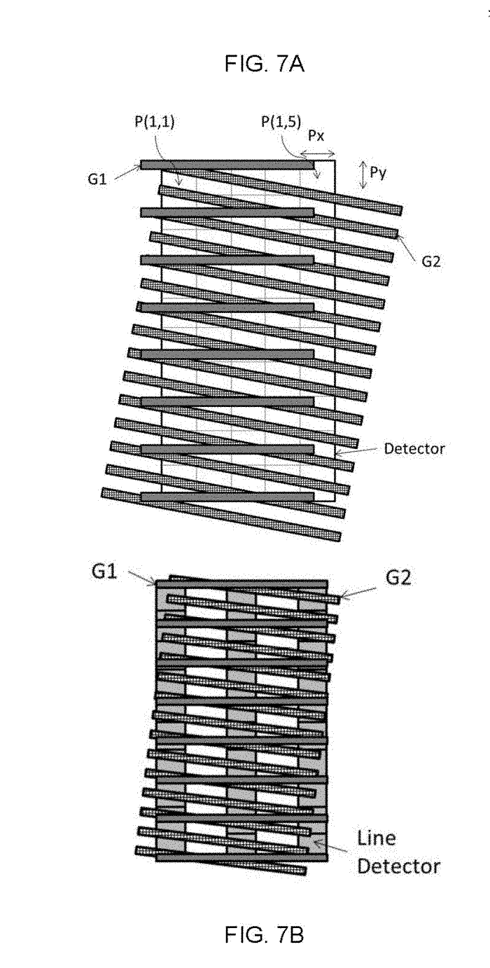

FIG. 7A is a schematic diagram that illustrates gratings G1 and G2 that are oriented such that the scan lines are parallel to the scan direction, and in which the gratings G2 and G1 are slightly tilted with respect to one another.

Referring to FIG. 7A, in which the tilt of G2 relative to G1 is exaggerated for illustration, pixels P(1,1) through P(1,5) each measure a different phase shift and the grating G2 is tilted such that the desired number of phase shifts covering integral multiples of 2π is achieved. As the object is scanned, assuming left to right motion in the figure, the object sampled by pixel P(1,5) that provides a measure corresponding to one phase shift is then sampled by pixel P(1,4) which provides a measure corresponding to a different phase shift. Thus, when all pixels have traversed the object, all of the required phase shift need to obtain differential phase images would have been obtained.

FIG. 7B is a schematic diagram similar to FIG. 7A, in which multiple line detectors are used instead of a two-dimensional pixel array detector.

Apparatus constructed and operated according to principles of the invention overcomes one current limitation of phase contrast measurement methods. The invention eliminates the need for phase stepping which, for applications seeking to make measurements having precision of the order of a micron or less requires a high precision moving assembly using the conventional prior art apparatus and methods. In addition, the invention provides the ability to use gratings of smaller size than are conventionally used that correspond to either the scanning detector assembly or the scanning detector sub-assembly. Depending on the direction of the grating lines relative to the scan direction, e.g., grating lines parallel or perpendicular to the scan direction, the size of the gratings will depend on either the scanning detector assembly or the scanning detector sub-assembly, respectively. In addition, the invention provides systems and methods for obtaining phase contrast images in addition to differential phase contrast images, by integrating the differential phase contrast images over the scan direction, provided the scan covers the entire object.

The invention is expected to have widespread applications in all x-ray imaging methods including, radiography, mammography, non-destructive testing, tomosynthesis and computed tomography.

### Exemplary Apparatus

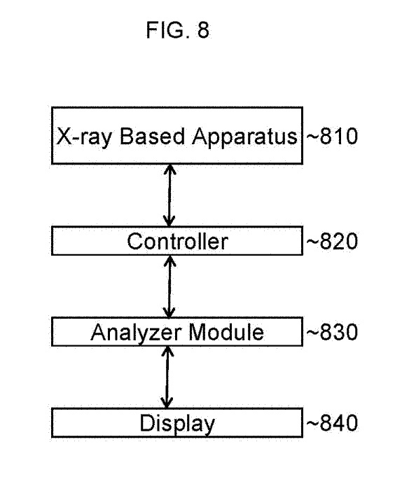

FIG. 8 is a schematic diagram that illustrates the components of an exemplary apparatus according to principles of the invention and the interactions among the components. As illustrated in FIG. 8, an x-ray-based apparatus 810 such as is shown in any of FIG. 2, FIG. 3, FIG. 4, FIG. 5, FIG. 6, or FIG. 7 is provided to perform the positioning of the x-ray source, the source grating, the phase grating, and the analyzer grating configured to move as a single entity relative to an object of interest to be examined. The x-ray source, the source grating, the phase grating, and the analyzer grating are configured to move as a single entity relative to an object of interest. A controller 820 is provided that communicates bi-directionally with the apparatus 810. The controller 820 controls the activities of the apparatus 810, and receives data from one or more x-ray sensitive detectors in the apparatus 810. An analyzer module 830 communicates with the controller 820, to direct the controller to control the apparatus 810, and to receive from the controller 820 data to be process to generate at least one phase contrast image of the object of interest. The analyzer module 830 is in one embodiment a general purpose programmable computer provided with instructions recorded on a machine readable medium, and includes a memory upon which the data and/or the generated images can be recorded. The analyzer module 830 communicates with a display 840, which can display one or more generated images to a user. The display can have one or more display screens, and can operate so as to provide a phase contrast x-ray image if and when such an image is provided for display. The analyzer module 830 also includes a user interface that permits a user to initiate operation of the apparatus, and permits a user to request that results be provided as any of a displayed image, a recorded image, recorded data, and data and/or images to be provided to a user at a remote location. In some embodiments, the controller 820, the analyzer module 830 and the display 840 are all part of the same general purpose programmable computer provided with instructions recorded on a machine readable medium.

### Definitions

Unless otherwise explicitly recited herein, any reference to an electronic signal or an electromagnetic signal (or their equivalents) is to be understood as referring to a non-volatile electronic signal or a non-volatile electromagnetic signal.

Recording the results from an operation or data acquisition, such as for example, recording results at a particular frequency or wavelength, is understood to mean and is defined herein as writing output data in a non-transitory manner to a storage element, to a machine-readable storage medium, or to a storage device. Non-transitory machine-readable storage media that can be used in the invention include electronic, magnetic and/or optical storage media, such as magnetic floppy disks and hard disks; a DVD drive, a CD drive that in some embodiments can employ DVD disks, any of CD-ROM disks (i.e., read-only optical storage disks), CD-R disks (i.e., write-once, read-many optical storage disks), and CD-RW disks (i.e., rewriteable optical storage disks); and electronic storage media, such as RAM, ROM, EPROM, Compact Flash cards, PCMCIA cards, or alternatively SD or SDIO memory; and the electronic components (e.g., floppy disk drive, DVD drive, CD/CD-R/CD-RW drive, or Compact Flash/PCMCIA/SD adapter) that accommodate and read from and/or write to the storage media. Unless otherwise explicitly recited, any reference herein to “record” or “recording” is understood to refer to a non-transitory record or a non-transitory recording.

As is known to those of skill in the machine-readable storage media arts, new media and formats for data storage are continually being devised, and any convenient, commercially available storage medium and corresponding read/write device that may become available in the future is likely to be appropriate for use, especially if it provides any of a greater storage capacity, a higher access speed, a smaller size, and a lower cost per bit of stored information. Well known older machine-readable media are also available for use under certain conditions, such as punched paper tape or cards, magnetic recording on tape or wire, optical or magnetic reading of printed characters (e.g., OCR and magnetically encoded symbols) and machine-readable symbols such as one and two dimensional bar codes. Recording image data for later use (e.g., writing an image to memory or to digital memory) can be performed to enable the use of the recorded information as output, as data for display to a user, or as data to be made available for later use. Such digital memory elements or chips can be standalone memory devices, or can be incorporated within a device of interest. “Writing output data” or “writing an image to memory” is defined herein as including writing transformed data to registers within a microcomputer.

“Microcomputer” is defined herein as synonymous with microprocessor, microcontroller, and digital signal processor (“DSP”). It is understood that memory used by the microcomputer, including for example instructions for data processing coded as “firmware” can reside in memory physically inside of a microcomputer chip or in memory external to the microcomputer or in a combination of internal and external memory. Similarly, analog signals can be digitized by a standalone analog to digital converter (“ADC”) or one or more ADCs or multiplexed ADC channels can reside within a microcomputer package. It is also understood that field programmable array (“FPGA”) chips or application specific integrated circuits (“ASIC”) chips can perform microcomputer functions, either in hardware logic, software emulation of a microcomputer, or by a combination of the two. Apparatus having any of the inventive features described herein can operate entirely on one microcomputer or can include more than one microcomputer.

General purpose programmable computers useful for controlling instrumentation, recording signals and analyzing signals or data according to the present description can be any of a personal computer (PC), a microprocessor based computer, a portable computer, or other type of processing device. The general purpose programmable computer typically comprises a central processing unit, a storage or memory unit that can record and read information and programs using machine-readable storage media, a communication terminal such as a wired communication device or a wireless communication device, an output device such as a display terminal, and an input device such as a keyboard. The display terminal can be a touch screen display, in which case it can function as both a display device and an input device. Different and/or additional input devices can be present such as a pointing device, such as a mouse or a joystick, and different or additional output devices can be present such as an enunciator, for example a speaker, a second display, or a printer. The computer can run any one of a variety of operating systems, such as for example, any one of several versions of Windows, or of MacOS, or of UNIX, or of Linux. Computational results obtained in the operation of the general purpose computer can be stored for later use, and/or can be displayed to a user. At the very least, each microprocessor-based general purpose computer has registers that store the results of each computational step within the microprocessor, which results are then commonly stored in cache memory for later use.

Many functions of electrical and electronic apparatus can be implemented in hardware (e.g., hard-wired logic), in software (e.g., logic encoded in a program operating on a general purpose processor), and in firmware (e.g., logic encoded in a non-volatile memory that is invoked for operation on a processor as required). The present invention contemplates the substitution of one implementation of hardware, firmware and software for another implementation of the equivalent functionality using a different one of hardware, firmware and software. To the extent that an implementation can be represented mathematically by a transfer function, that is, a specified response is generated at an output terminal for a specific excitation applied to an input terminal of a “black box” exhibiting the transfer function, any implementation of the transfer function, including any combination of hardware, firmware and software implementations of portions or segments of the transfer function, is contemplated herein, so long as at least some of the implementation is performed in hardware.

Although the theoretical description given herein is thought to be correct, the operation of the devices described and claimed herein does not depend upon the accuracy or validity of the theoretical description. That is, later theoretical developments that may explain the observed results on a basis different from the theory presented herein will not detract from the inventions described herein.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

What is claimed is:

1. An x-ray phase contrast imaging apparatus, comprising:
an x-ray source configured to provide x-ray illumination at an exit port thereof;
a source grating configured to receive said x-ray illumination at a source grating entrance port and configured to provide a plurality of x-ray beams at a source grating exit port;
a phase grating having a plurality of phase grating elements, said phase grating situated at a distance l from said source grating, said phase grating configured to receive x-rays at a phase grating entrance port and to provide x-rays at a phase grating exit port;
an analyzer grating having a plurality of analyzer grating elements, said analyzer grating situated at a distance d from said phase grating, said phase grating and said analyzer grating are disposed such that the locations and orientations of said phase grating and said analyzer grating are fixed relative to each other and do not move relative to each other during operation of the apparatus, said analyzer grating configured to receive x-rays at a analyzer grating entrance port and to provide x-rays at a analyzer grating exit port;
said x-ray source, said source grating, said phase grating, and said analyzer grating configured to move as a single entity relative to an object of interest;
an x-ray sensitive detector comprising a two-dimensional array detector with a plurality of pixels oriented in two-dimensions, which detector is positioned so as to receive x-rays generated by said x-ray source after said x-rays have passed sequentially through said source grating, through said object of interest, through said phase grating and through said analyzer grating, said x-ray-sensitive detector having at least one output terminal configured to provide electrical signals representative of said received x-rays;
a controller configured to control the motion of said x-ray source, said source grating, said phase grating, and said analyzer grating relative to said object of interest as a function of time, and configured to control said x-ray source and said x-ray sensitive detector as a function of time; and
an analyzer module configured to receive and record said electrical signals representative of said received x-rays as a function of time, configured to manipulate said received electrical signals with respect to time, configured to generate a phase contrast image of at least a portion of said object of interest said from received electrical signals, and configured to perform at least one action selected from the group of actions consisting of recording said x-ray phase contrast image, transmitting said x-ray phase contrast image to a data handling system, and displaying said x-ray phase contrast image to a user.

2. The x-ray phase contrast imaging apparatus of claim 1, wherein said x-ray source is a conventional x-ray tube with an x-ray focal spot.

3. The x-ray phase contrast imaging apparatus of claim 1, wherein said x-ray source is a hot filament x-ray source.

4. The x-ray phase contrast imaging apparatus of claim 1, further comprising an object support configured to support said object of interest.

5. The x-ray phase contrast imaging apparatus of claim 4, further comprising a compression paddle.

6. (canceled)

7. The x-ray phase contrast imaging apparatus of claim 1, wherein said analyzer module is a general purpose programmable computer provided with instructions recorded on a machine readable medium.

8. The x-ray phase contrast imaging apparatus of claim 1, wherein said controller and said analyzer module are each part of a single general purpose programmable computer provided with instructions recorded on a machine readable medium.

9. The x-ray phase contrast imaging apparatus of claim 1, wherein said source grating is configured to provide a plurality of x-ray beams that individually exhibit spatial coherence at said source grating exit port.

10. The x-ray phase contrast imaging apparatus of claim 1, wherein said phase grating having a plurality of phase grating elements and said analyzer grating having a plurality of analyzer grating elements are configured such that a first phase shift is provided between a first of said plurality of phase grating elements and a first of said plurality of analyzer grating elements, a second phase shift different from the first phase shift is provided between a second of said plurality of phase grating elements and a second of said plurality of analyzer grating elements, and at least one additional phase shift is provided by a third of said plurality of phase grating elements and a third of said plurality of analyzer grating elements.

11. A method of making an x-ray phase contrast image of an object of interest, comprising the steps of:
passing x-rays generated by an x-ray source through, in succession, a source grating, an object of interest, a phase grating, and a analyzer grating while causing said x-ray source, said source grating, said phase grating, and said analyzer grating to move as a single entity relative to an object of interest, said phase grating and said analyzer grating remaining in fixed relative location and fixed relative orientation with respect to one another;
detecting transmitted x-rays with an x-ray sensitive detector comprising a two-dimensional array detector with a plurality of pixels oriented in two-dimensions, said x-ray sensitive detector providing electrical signals representative of said detected x-rays as output signals, wherein said phase grating and said analyzer grating do not move relative to each other during the above steps;
analyzing said electrical signals representative of said detected x-rays as a function of time to generate an x-ray phase contrast image of said object of interest; and
performing at least one action selected from the group of actions consisting of recording said x-ray phase contrast image, transmitting said x-ray phase contrast image to a data handling system, and displaying said x-ray phase contrast image to a user,
wherein said x-ray source is a conventional x-ray tube with an x-ray focal spot.

12. The method of claim 11, wherein each of said source grating, said phase grating and said analyzer grating comprises grating lines that are oriented orthogonal to a scan direction of said x-ray beams.

13. The method of claim 11, wherein each of said source grating, said phase grating and said analyzer grating comprises grating lines that are oriented parallel to a scan direction of said x-ray beams.

14. A method of making an x-ray phase contrast image of an object of interest, comprising the steps of:
passing x-rays generated by an x-ray source through, in succession, a source grating, an object of interest, a phase grating, and a analyzer grating while causing said x-ray source, said source grating, said phase grating, and said analyzer grating to move as a single entity relative to an object of interest, said phase grating and said analyzer grating remaining in fixed relative location and fixed relative orientation with respect to one another;
detecting transmitted x-rays with an x-ray sensitive detector comprising a two-dimensional array detector with a plurality of pixels oriented in two-dimensions, said x-ray sensitive detector providing electrical signals representative of said detected x-rays as output signals, wherein said phase grating and said analyzer grating do not move relative to each other during the above steps;
analyzing said electrical signals representative of said detected x-rays as a function of time to generate an x-ray phase contrast image of said object of interest; and
performing at least one action selected from the group of actions consisting of recording said x-ray phase contrast image, transmitting said x-ray phase contrast image to a data handling system, and displaying said x-ray phase contrast image to a user,
wherein said x-ray source is a field emission x-ray source.

15. The method of claim 14, wherein each of said source grating, said phase grating and said analyzer grating comprises grating lines that are oriented orthogonal to a scan direction of said x-ray beams.

16. The method of claim 14, wherein each of said source grating, said phase grating and said analyzer grating comprises grating lines that are oriented parallel to a scan direction of said x-ray beams.

17. The x-ray phase contrast imaging apparatus of claim 1, wherein said x-ray source is a field emission x-ray source.

18. The x-ray phase contrast imaging apparatus of claim 17, wherein each of said source grating, said phase grating and said analyzer grating comprises grating lines that are oriented orthogonal to a scan direction of said x-ray beams.

19. The x-ray phase contrast imaging apparatus of claim 17, wherein each of said source grating, said phase grating and said analyzer grating comprises grating lines that are oriented parallel to a scan direction of said x-ray beams.
